(19) United States
(12) Patent Application Publication
(10) Pub. No.: US 2026/0311807 A1
(43) Pub. Date: Oct. 8, 2026

(54) IMMUNOLOGICAL ENTITY SEQUENCE DATA PROCESSING

(71) Applicant: Omniscope Limited, Mosta (MT)

(72) Inventors: Dmytro Pravdyvets, Mosta (MT); Ana Mendizabal Sasieta, Mosta (MT)

(21) Appl. No.: 19/482,605

(22) PCT Filed: May 9, 2024
(86) PCT No.: PCT/EP2024/062863

§ 371 Date: Nov. 7, 2025

(30) Foreign Application Priority Data
May 9, 2023 (GB) 2306811.7

(51) Int. Cl. G16B 30/00; G16B 40/10; G16B 40/30

(52) U.S. Cl. CPC G16B 30/00; G16B 40/10; G16B 40/30

(57) ABSTRACT

Immunological entity sequences are clustered based on their similarity i.e. ability to react to the same antigens, by a two-stage process. In a first stage, the sequences are converted into numerical vectors which are clustered into initial clusters. In a second stage, the initial clusters are refined into final clusters, which may be done using the sequence data itself rather than the numerical vectors. Each final cluster may be assigned a representative sequence so as to reduce the dimensionality of the clusters.

### TECHNICAL FIELD

The present invention relates to a method and computer program for processing sequence data from immunological entities, such as T cell receptor (TCR) or B cell receptor (BCR) sequence data.

### BACKGROUND ART

The T-cell receptor (TCR) is a protein complex found on the surface of T cells that is responsible for recognizing fragments of antigen peptides. The binding between TCR and antigen peptides is of relatively low affinity and is degenerate: many TCRs recognize the same antigen peptide and many antigen peptides are recognized by the same TCR. It is therefore desirable to be able to identify similar TCR sequences, for example based on their ability to react to the same or similar antigen peptides.

The TCR protein is composed of two chains, called the alpha chain and the beta chain, which are held together by disulphide bonds. Each chain contains variable (V), diversity (D), and joining (J) regions, which are responsible for the antigen recognition. The V regions of the alpha and beta chains come together to form the antigen-binding site or the complementarity-determining region (CDR), which is responsible for specifically recognizing the antigen.

The diversity of the TCR structure is made possible by the diversity of the amino acids that make up the V, D, and J regions of the alpha and beta chains. These regions contain a high variability of amino acid sequences, allowing for a wide range of antigen recognition. The amino acids in the CDRs of the TCR chains form a three-dimensional structure that enables the TCR to specifically recognize and bind to the antigen.

The sequence encoded by the V (D) J junction is called complementarity determining region 3 or CDR3. This sequence has the highest variability in both alpha and beta chains and determines the ability of a T cell to recognize an antigen peptide.

The different amino acids have distinct properties, such as size, charge, and polarity, which play a critical role in determining the structure and function of the TCR. For example, amino acids with large side chains, such as arginine or lysine, may be involved in forming salt bridges or hydrogen bonds, while amino acids with small side chains, such as glycine or alanine, may contribute to the flexibility of the TCR structure.

Reference NPL1 summarises repertoire sequencing technologies which were available for the study of B cell and T cell repertoire in 2012. Next generation immunosequencing technology, such as the OmniScope T Cell Sequencing Platform OS-T, are able to sequence up to 1 million T cells per sample, characterising immune response with a sensitivity that allows changes in repertories to be detected early and at high-resolution for advanced translational and clinical research. However, improved techniques are required to process and analyse the resulting large quantity of sequence data.

### SUMMARY OF INVENTION

According to an aspect of the invention, there is provided a method of processing cell sequencing data to produce a data structure that is suitable for downstream analysis, by clustering based on similarity e.g. the ability to react to the same antigen or antigens.

The clustering may be performed by a two-stage process. In a first stage, the sequences are converted into numerical vectors which are clustered into initial clusters. In a second stage, the initial clusters are refined into final clusters, which may be done using the sequence data itself rather than the numerical vectors.

The dimensionality of the sequence data may be reduced so as to preserve useful characteristics for further analysis while discarding redundant information. In this case, the useful characteristics may be representative of variability of the immune repertoire. To achieve this, each cluster may be assigned a representative sequence. The representative sequence may be used as a representation of a set of N sequences within the corresponding cluster, which can be used to simplify any downstream analysis without losing the diversity of TCR repertoire.

The resultant output of the method may comprise a set of clusters each containing sequences of high sequence similarity and likely similar functionality. Each cluster can be analysed individually to precisely identify similar sequences in the repertoire or the entire set of clusters can be used to analyze the density, sparsity and other metrics of a TCR repertoire.

### BRIEF DESCRIPTION OF DRAWINGS

Specific embodiments will now be described with reference to the accompanying drawings as identified below.

FIG. 1 is a flowchart illustrating a method of processing TCR sequence data in an embodiment of the invention.

FIG. 2 shows an example of score values in a BLOSUM62 matrix.

FIG. 3 is a diagram illustrating an example of a computer system which may execute the method.

### DESCRIPTION OF EMBODIMENTS

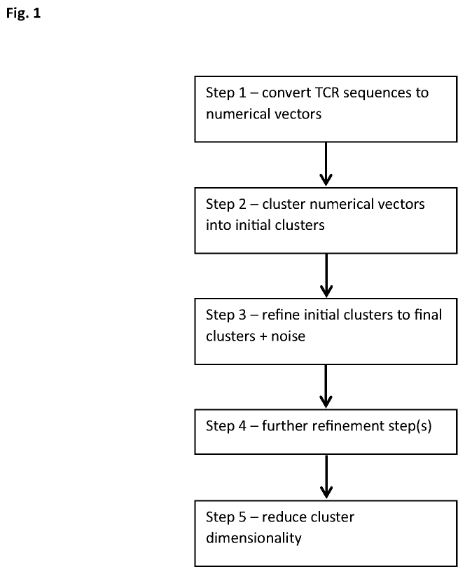

As illustrated in FIG. 1, a method in one embodiment comprises the following steps:
1. Each TCR sequence is transferred into a numerical vector, for example using a Natural Language Processing (NLP) model or by embedding evolutionary or structural properties of the sequence.
2. The TCR sequence vectors are clustered into initial superclusters, for example using a fast K-means clustering algorithm.
3. The clusters are refined to create final clusters and a noise class.
4. Further clustering refinement step(s)
5. Reduce cluster dimensionality

Steps 4 and/or 5 above may be optional, depending on the application of the method. Each of these steps is explained in more detail below.

The TCR sequences may comprise CDR3 sequence data representing CDR3 sequences from a TCR repertoire. Each item of CDR3 sequence data may comprise a string, examples of which are given below:

SEQ1
CASSPSGTPYEQYF
SEQ2
CASSPSGTTTPYEQYF
SEQ3
CASSPSGGTTPYEQYF
SEQ4
ASSQASQQQQF
SEQ5
ASSSQQQQF
SEQ6
QQWQQQSAPA
SEQ7
SARAPPYPYNEQF

The above are a small number of example sequences, however the method is applicable to processing very large numbers of sequences.

In Step 1, each item of TCR sequence data is transformed into a corresponding numerical vector for efficient processing. This can be done in many different ways. One example is SeqVec, based on an NLP (Natural Language Processing) model that uses ELMo (Embeddings from Language Models), as described for example in reference NPL2. This model transfers the sequences into a 1024-dimensional vector that contains their amino acid biophysical information and can be used for downstream data processing. Another NLP model that may be used is a masked-language model such as BERT (Bidirectional Encoder Representations from Transformers), as described for example in reference NPL7.

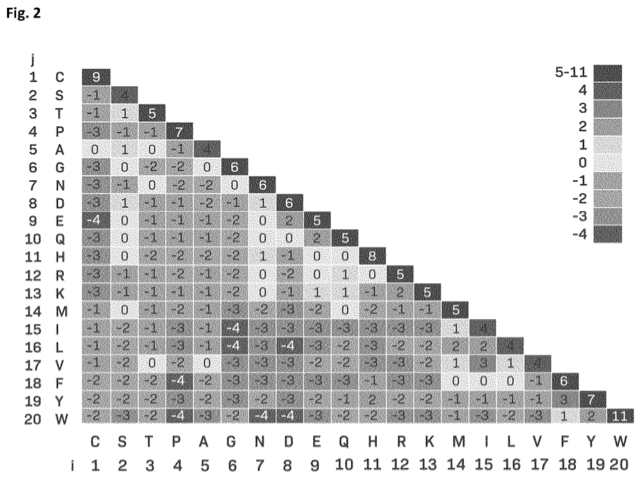

An alternative approach in Step 1 is to use matrix multiplication. Two matrices are chosen as the value matrices. The first value matrix may be a BLOSUM (BLOcks Substitution Matrix), as disclosed for example in reference NPL3. BLOSUM assigns a score for the substitution of the ith amino acid with the jth amino acid. The score values may be those of BLOSUM62, based on a threshold identity of 62% or less, as shown for example in [FIG. 2].

The second value matrix may be a Miyazawa matrix, as disclosed for example in reference NPL4. By multiplying these matrices, an output matrix may be generated of the same size even for sequences of different length. Since both BLOSUM62 and Miyazawa matrices contain 21 values per amino acid, the result is a vector of size 441. This vector is then used as input for the next step.

In the example of SEQ1 above, the corresponding numerical vector is a one-dimensional vector with values in the range −1 to +1, such as [0.8, −0.2 . . . 0.1, −0.73].

In Step 2, the numerical vectors representing the sequences are clustered together. To process the large quantity of data efficiently, the clustering is split into multiple stages. In the first stage, the vectors are clustered into initial ‘superclusters’ which will later be split into final clusters. The first stage may be performed using a K-means clustering algorithm such as FAISS (documented at https://faiss.ai/), which provides an approximate solution which is sufficient for the initial clustering.

In the sequence examples given above, the initial clusters may be defined as follows:
SC1: SEQ1-3
SC2: SEQ4-6
SC3: SEQ7

In Step 3, the initial clusters are refined to form final clusters, for example using HDBSCAN (see reference NPL5), which is a density-based algorithm expected to find some sequence motifs with higher density (similar sequences) compared to others based on the underlying signal in the sample. For this step, clustering is performed on the sequence data i.e. the sequence data strings provided as input at step 1, rather than the corresponding numeric vectors generated at step 1 and clustered in step 2.

The clustering of amino acid sequences may be clustered using Levenshtein distance (see reference NPL6), which indicates how many edits must be made for one string to become another string. Amino acid sequences with edit distance of 1-2 are expected to maintain a very similar functionality and binding affinity to epitopes.

Thus by using a distance matrix between the receptors as the input to HDBSCAN, clusters of very similar sequences are obtained both on functional level (from numerical vectors in step 2) and sequence identity wise (using Levenshtein distance in step 3).

A density-based definition may be used for the final clusters. This allows the method to work with clusters of different size and sparsity, which downstream accounts both for clusters of high density (big number of similar receptors) and unique receptors that are unique in the repertoire.

In the case that a sequence is not assigned to any cluster in Step 3, it is assigned to a Noise Class (NC), which contains all the sequences that were either:
a) misassigned to a wrong supercluster so they did not end up with the similar sequences or
b) very different sequences from the rest of the repertoire.

In the sequence examples given above, the final clusters may be defined as follows:
FC1: SEQ1-3
FC2: SEQ4-5
FC3: SEQ6
NC: SEQ7

Additional refinement steps may be performed after step 3. For example, at step 4 all the sequences assigned to the Noise class are processed again using steps 2 and 3, to generate new clusters that are added to the final clusters. Sequences that are still in the Noise class at this point are defined as clusters of size 1. As an alternative or additional refinement step 4, step 3 (density-based clustering) may be performed again on the final clusters, including any new clusters.

As an alternative or additional refinement step 4, clusters with a high (e.g. above a predetermined threshold) average pairwise Levenshtein distance may be dissolved i.e. the member sequences are removed from the cluster and the cluster is deleted.

One or more of the refinement steps may be performed iteratively until one or more criteria are met e.g. minimal or no change in clustering after an iteration.

The output of the method after step 3 (and optionally step 4) may be a data file containing a set of N clusters each containing sequences of high sequence similarity and likely functional similarity. These may be analysed as explained further below, but optionally the data representing each final cluster may be reduced as set out below.

At step 5, the dimensionality of each final cluster FC is reduced, for example by selecting a representative sequence (or sequences) from each final cluster. The representative sequence may then be used for later analysis, instead of analysing all the sequences from each final cluster.

In one option, the most expanded sequence of each final cluster is selected as the representative sequence of that cluster. If there are a plurality of sequences with the same maximum expansion value, one of the plurality of sequences is selected as the representative sequence. For example, the first of the plurality of sequences to be added to the final cluster is selected as the representative sequence.

In an alternative option, the selection of the representative sequence is based on string distance. A matrix with each pair of Levenshtein distances is calculated. The total distance of each sequence is calculated and the sequence with the smallest total distance is selected as the representative distance. If there are a plurality of sequences with the same smallest total distance, one of the plurality of sequences is selected as the representative sequence. For example, the first of the plurality of sequences to be added to the final cluster is selected as the representative sequence.

### Potential Applications

The resulting dataset of sequences and the respective clusters, optionally with reduced dimensionality as explained above, have many potential uses in research, diagnostics and/or therapeutics, examples of which are set out below.

The dataset may be used for prediction and/or classification in clinical scenarios. For example, the clusters may be indicative of exposure to particular pathogens or may be used to discover new antigens which may be used in disease-specific drug development.

The resulting dataset of sequences and the respective clusters, optionally together with descriptive data and/or reduced dimensionality as explained above, can also be used for other purposes such as a sequence query. Using the Levenshtein distance, one can retrieve a specific sequence or a similar motif that is present in a sample. This allows for quick search for a signal from sample to sample that can help to define public responses to specific antigens.

Vaccine development: TCR CDR3 clusters can be used to design and optimize vaccines. By identifying TCR CDR3 clusters that are associated with protective immune responses against certain pathogens, researchers can develop vaccines that elicit targeted immune responses and enhance vaccine efficacy.

Diagnostics: TCR CDR3 clusters can be used as diagnostic markers for diseases associated with aberrant immune responses, such as autoimmune diseases or cancer. The presence or absence of specific TCR CDR3 clusters in patient samples can be used to diagnose disease conditions, monitor disease progression, or assess response to immunotherapies.

Therapeutics: TCR CDR3 clusters can be used in the development of TCR-based immunotherapies, such as TCR-engineered T cell therapies. TCR CDR3 clusters with high affinity and specificity for tumour antigens or viral antigens can be incorporated into T cells to create genetically modified T cells that can recognize and eliminate cancer cells or infected cells.

Immune repertoire profiling: TCR CDR3 clusters can be used for profiling the diversity and dynamics of the immune repertoire in response to different diseases, infections, or treatments. This can provide valuable information about the clonal diversity, expansion, and contraction of T cell populations, which can aid in understanding immune responses and developing immunotherapies.

Drug discovery: TCR CDR3 clusters can be used in drug discovery efforts to develop small molecules or biologics that specifically target TCR-mediated immune responses. By understanding the interactions between TCR CDR3 clusters and their cognate antigens, researchers can design therapeutics that modulate TCR signalling or function.

The above are just a few potential uses of TCR CDR3 clusters of high quality. The field of TCR research is rapidly evolving, and further applications and discoveries are likely to emerge as our understanding of TCR biology and immunology advances.

### Computer System

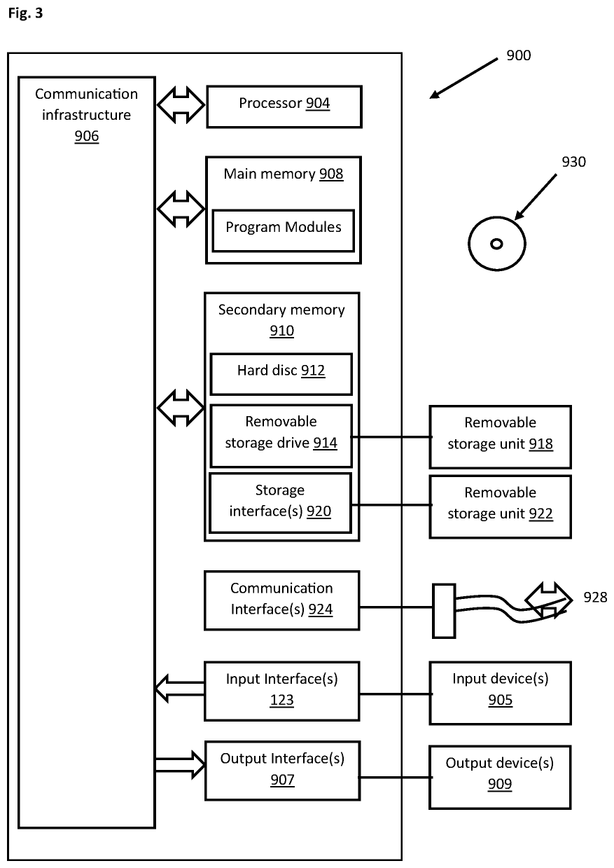

FIG. 3 illustrates an example computer system 900 in which the present invention, or portions thereof, can be implemented as computer-readable code to program processing components of the computer system 900. Various embodiments of the invention are described in terms of this example computer system 900. For example, the steps of the method of FIG. 1 can each be implemented in system 900. After reading this description, it will become apparent to a person skilled in the relevant art how to implement the invention using other computer systems and/or computer architectures.

Computer system 900 includes one or more processors, such as processor 904. Processor 904 can be a special purpose or a general-purpose processor. Processor 904 is connected to a communication infrastructure 906 (for example, a bus, or network). Computer system 900 also includes a user input interface 903 connected to one or more input device(s) 905 and a display interface 907 connected to one or more output devices 909, such as a display, which may be integrated input and display components. Input devices 905 may include, for example, a connected device such as a mouse or touchpad, a keyboard, a touchscreen such as a resistive or capacitive touchscreen, etc.

Computer system 900 also includes a main memory 908, preferably random-access memory (RAM), and may also include a secondary memory 910. Secondary memory 910 may include, for example, a hard disk drive 912, a removable storage drive 914, flash memory, a memory stick, and/or any similar non-volatile storage mechanism. Removable storage drive 914 may comprise a floppy disk drive, a magnetic tape drive, an optical disk drive, a flash memory, or the like. The removable storage drive 914 reads from and/or writes to a removable storage unit 918 in a well-known manner. Removable storage unit 918 may comprise a floppy disk, magnetic tape, optical disk, etc. which is read by and written to by removable storage drive 914. As will be appreciated by persons skilled in the relevant art(s), removable storage unit 918 includes a non-transitory computer usable storage medium having stored therein computer software and/or data.

In alternative implementations, secondary memory 910 may include other similar means for allowing computer programs or other instructions to be loaded into computer system 900. Such means may include, for example, a removable storage unit 922 and an interface 920. Examples of such means may include a program cartridge and cartridge interface (such as that found in video game devices), a removable memory chip (such as an EPROM, or PROM) and associated socket, and other removable storage units 922 and interfaces 920 which allow software and data to be transferred from the removable storage unit 922 to computer system 900.

Computer system 900 may also include a communications interface 924 implemented for example at the operating system level to allow data to be transferred between computer system 900 and external devices, for example as signals 928 over a communication channel 926. Communications interface 924 may include a modem, a network interface (such as an Ethernet card), a communications port, a PCMCIA slot and card, or the like.

Various aspects of the present invention, such as one or more of the method steps described above, can be implemented by software and/or firmware (also called computer programs, instructions or computer control logic) to program programmable hardware, or hardware including special-purpose hardwired circuits such as application-specific integrated circuits (ASICs), programmable logic devices (PLDs), field-programmable gate arrays (FPGAs), etc. of the computer system 900, or a combination thereof. Computer programs for use in implementing the techniques introduced here may be stored on a machine-readable storage medium and may be executed by one or more general-purpose or special-purpose programmable microprocessors. The terms “computer program medium”, “non-transitory computer readable medium” and “computer usable medium” introduced herein can generally refer to media such as removable storage unit 918, removable storage unit 922, and a hard disk installed in hard disk drive 912. Computer program medium, computer readable storage medium, and computer usable medium can also refer to memories, such as main memory 908 and secondary memory 910, which can be memory semiconductors (e.g. DRAMs, etc.). These computer program products are means for providing software to computer system 900.

Computer programs are stored in main memory 908 and/or secondary memory 910.

Computer programs may also be received via communications interface 924. Such computer programs, when executed, enable computer system 900 to implement the present invention as described herein. In particular, the computer programs, when executed, enable processor 904 to implement the processes of embodiments of the present invention as described above. Accordingly, such computer programs represent controllers of the computer system 900. Where the invention is implemented using software, the software may be stored in a computer program product and loaded into computer system 900 using removable storage drive 914, interface 920, hard drive 912, or communications interface 924.

### Alternative Embodiments

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as an “engine”, a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Aspects of the present disclosure are described above with reference to flowchart and/or block diagrams of methods, apparatus (systems) and computer program products according to embodiments of the disclosure. It will be understood that each block of the flowchart and/or block diagrams, and combinations of blocks in the flowchart and/or block diagrams, can be implemented by computer program instructions. These computer program instructions may be provided to a processor of a general-purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the blocks of the flowchart diagrams may occur out of the order noted in the figures. For example, two blocks shown in succession may, in fact, be executed substantially concurrently, or the blocks may sometimes be executed in the reverse order, depending upon the functionality involved. It will also be noted that each block of the flowchart diagrams, and combinations of blocks in the flowchart diagrams, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

### NON-PATENT LITERATURE

NPL1: ‘Rep-Seq: uncovering the immunological repertoire through next-generation sequencing’, Benichou et al. Immunology. 2012 March; 135 (3): 183-191
NPL2: ‘Modeling aspects of the language of life through transfer-learning protein sequences’ Heinzinger et al. BMC Bioinformatics (2019) 20:723
NPL3: ‘Amino acid substitution matrix from protein blocks’, Henikoff S, Henikoff J G, Proc Natl Acad Sci USA 89:10915-10919 (1992)
NPL4: ‘Estimation of effective interresidue contact energies from protein crystal structures: quasi-chemical approximation’, Miyazawa S, Jernigan R, Macromolecules 1985, 18, 3, 534-552.
NPL5: ‘Density-Based Clustering Based on Hierarchical Density Estimates’, Campello, R. J. G. B., Moulavi, D., Sander, J. (2013. In: Pei, J., Tseng, V. S., Cao, L., Motoda, H., Xu, G. (eds) Advances in Knowledge Discovery and Data Mining. PAKDD 2013. Lecture Notes in Computer Science ( ) vol 7819. Springer, Berlin, Heidelberg
NPL6: ‘Binary Codes Capable of Correcting Deletions, Insertions and Reversals; Levenshtein V, Soviet Physics-Doklady, Vo. 10, no. 8, February 1966
NPL7: “BERT: Pre-training of Deep Bidirectional Transformers for Language Understanding”; Devlin, Jacob; Chang, Ming-Wei; Lee, Kenton; Toutanova, Kristina (11 Oct. 2018)

1. A method of processing TCR or BCR cell sequence data, comprising:
accessing a plurality of cell sequence data items, each item representing a complementarity-determining region (CDR) of a TCR or BCR cell sequence;
converting the cell sequence data items into corresponding numerical vectors;
clustering the numerical vectors into a plurality of initial clusters;
refining the initial clusters based on the corresponding cell sequence data items, to generate final clusters; and
outputting clustering data representing the final clusters.

2. The method of claim 1, wherein the cell sequence data items are converted into corresponding numerical vectors by multiplication of first and second value matrices.

3. The method of claim 2, wherein the first value matrix is a BLOSUM matrix.

4. The method of claim 2, wherein the second value matrix is a Miyazawa matrix.

5. The method of claim 1, wherein the numerical vectors are clustered into the plurality of initial clusters using a K-means clustering algorithm.

6. The method of claim 1, wherein any of said numerical vectors that cannot be clustered into one of said initial clusters are assigned to a noise class.

7. The method of claim 6, wherein refining the initial clusters includes assigning one or more cell sequence data items, which were assigned to the noise class, to one or more of said refined clusters.

8. The method of claim 1, wherein the initial clusters are refined using a density-based clustering algorithm.

9. The method of claim 8, wherein the density-based clustering algorithm is based on Levenshtein distances between pairs of cell sequence data items.

10. The method of claim 1, wherein said refining includes processing any cell sequence data items remaining in the noise class using said clustering and refining steps.

11. The method of claim 1, wherein said refining includes iterative clustering.

12. The method of claim 1, wherein said refining includes dissolving any clusters having an average pairwise distance above a predetermined threshold.

13. The method of claim 1, wherein said output clustering data includes a respective selected sequence data item from each said final cluster.

14. The method of claim 13, wherein said respective sequence data item is selected as having a maximum expansion value among the sequence data items within the corresponding final cluster.

15. The method of claim 13, wherein said respective sequence data item is selected as having a minimum total pairwise distance among the sequence data items within the corresponding final cluster.

16. A system for processing TCR or BCR cell sequence data, the system comprising:
a memory arranged to store a plurality of cell sequence data items, each item representing a complementarity-determining region (CDR) of a TCR or BCR cell sequence; and
a processor arranged to:
convert the cell sequence data items into corresponding numerical vectors;
cluster the numerical vectors into a plurality of initial clusters;
refine the initial clusters based on the corresponding cell sequence data items, to generate final clusters; and
output clustering data representing the final clusters.

17. A non-transitory computer-readable storage medium comprising instructions which, when executed by a processor of a computer, cause the computer processor to:
access a plurality of cell sequence data items, each item representing a complementarity-determining region (CDR) of a TCR or BCR cell sequence;
convert the cell sequence data items into corresponding numerical vectors;
cluster the numerical vectors into a plurality of initial clusters;
refine the initial clusters based on the corresponding cell sequence data items, to generate final clusters; and
output clustering data representing the final clusters.
